(19) United States
(12) Patent Application Publication
(10) Pub. No.: US 2024/0150341 A1
(43) Pub. Date: May 9, 2024

(54) COMPOSITION FOR PREVENTING OR TREATING DISEASES CAUSED BY MITOCHONDRIAL DYSFUNCTION, CONTAINING ISOQUINOLINE DERIVATIVE COMPOUND AS ACTIVE INGREDIENT

(71) Applicant: ALTMEDICALCO., LTD., Seoul (KR)

(72) Inventors: Jean Ho YUN, Busan (KR); Jong Hyun CHO, Gyeongsangnam-do (KR); Jee Hyun UM, Busan (KR); Dong Jin SHIN, Busan (KR); Se Myeong CHOI, Busan (KR)

(21) Appl. No.: 18/556,746

(22) PCT Filed: Sep. 3, 2021
(86) PCT No.: PCT/KR2021/011911

§ 371 Date: Oct. 23, 2023

(30) Foreign Application Priority Data
Apr. 23, 2021 (KR) 10-2021-0053069

(51) Int. Cl. C07D 455/03; A61K 9/00; A61K 9/20; A61K 9/48; A61K 47/02; A61K 47/10; A61K 47/12; A61K 47/14; A61K 47/20; A61K 47/22; A61K 47/26; A61K 47/44; A61K 47/46; A61P 25/28; A61P 43/00

(52) U.S. Cl. CPC C07D 455/03; A61K 9/0019; A61K 9/0043; A61K 9/0056; A61K 9/0095; A61K 9/2013; A61K 9/2018; A61K 9/2059; A61K 9/4858; A61K 9/4866; A61K 47/02; A61K 47/10; A61K 47/12; A61K 47/14; A61K 47/20; A61K 47/22; A61K 47/26; A61K 47/44; A61K 47/46; A61P 25/28; A61P 43/00

(57) ABSTRACT

The present invention relates to a composition for preventing or treating diseases caused by mitochondrial dysfunction, containing, as an active ingredient, an isoquinoline derivative compound represented by chemical formula 1, or a pharmaceutically acceptable salt thereof, and does not induce mitochondrial damage, unlike conventional mitochondrial toxins such as CCCP, and specifically and excellently promotes the activity of mitophagy to alleviate mitochondrial disfunction, and thus can be effectively used in the treatment of diseases caused by mitochondrial dysfunction.

### TECHNICAL FIELD

The present invention relates to a health food composition for preventing or improving a disease caused by mitochondrial dysfunction, comprising an isoquinoline derivative compound as an active ingredient.

### BACKGROUND ART

Mitophagy is a cellular degradation mechanism that eliminates damaged or unnecessary mitochondria. When mitochondrial damage occurs, it forms an autophagosome by surrounding it with a membrane and, by fusing this with a lysosome, selectively removes the damaged mitochondria. The activity of such mitophagy is known to be crucial in regulating mitochondrial function in various cells, including neurons, and in maintaining tissue function.

According to recent research, inhibition of mitophagy activity can lead to the accumulation of damaged mitochondria, inducing the death of motor neurons and potentially causing degenerative brain diseases like Alzheimer's. Furthermore, abnormalities in mitophagy activity have been reported to be related to a wide range of human diseases, including degenerative brain diseases such as Parkinson's, Alzheimer's, and Lou Gehrig's disease, as well as peripheral neuropathy, heart diseases, metabolic diseases, and cancer, heightening researchers' interest in the role of mitophagy in human diseases and its potential therapeutic applications.

Currently, experimental methods to induce mitophagy activity involve treating eith so-called ‘mitochondrial toxins’, such as CCCP, FCCP, and rotenone, which induce mitochondrial dysfunction. However, the CCCP and FCCP depolarize the mitochondrial membrane potential as uncouplers, and rotenone acts as a Complex I inhibitor. These mitochondrial toxins induce mitophagy activity by directly causing mitochondrial damage, but their strong toxicity to cells limits their use as drugs to promote mitophagy activity.

There is a genuine need for the development of a new pharmaceutical composition that effectively induces the activity of mitophagy without inducing mitochondrial dysfunction and is effective in preventing and treating Alzheimer's disease.

### DISCLOSURE

### Technical Problem

The present invention is directed to providing a health food composition for preventing or improving diseases caused by mitochondrial dysfunction, comprising an isoquinoline derivative compound represented by the following Chemical Formula 1 or its pharmaceutically acceptable salt as an active ingredient:

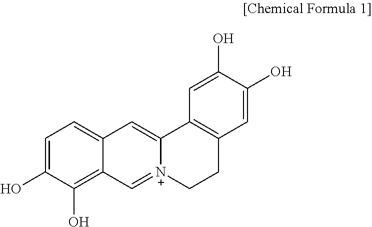 [SMILES: Oc1cc2c(cc1O)-c1cc3ccc(O)c(O)c3c[n+]1CC2]

The present invention is also directed to providing a method of preparing the isoquinoline derivative compound or a pharmaceutically acceptable salt thereof

Moreover, the present invention is directed to providing a health functional food composition for preventing or improving diseases caused by mitochondrial dysfunction, comprising the isoquinoline derivative compound or a pharmaceutically acceptable salt thereof as an active ingredient.

Furthermore, the present invention is directed to providing a pharmaceutical composition for preventing or treating diseases caused by mitochondrial dysfunction, comprising the isoquinoline derivative compound or a pharmaceutically acceptable salt thereof as an active ingredient.

### Technical Solution

To achieve the purposes of the present invention, the present invention provides a health food composition for preventing or improving diseases caused by mitochondrial dysfunction, wherein the composition comprises an isoquinoline derivative compound represented by the following Chemical Formula 1 or a pharmaceutically acceptable salt thereof as an active ingredient:

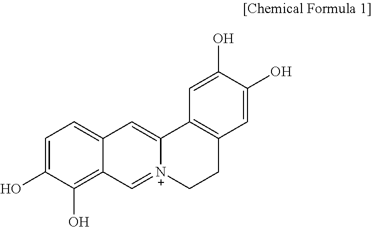 [SMILES: Oc1cc2c(cc1O)-c1cc3ccc(O)c(O)c3c[n+]1CC2]

Moreover, the present invention provides a method for preparing the isoquinoline derivative compound or a pharmaceutically acceptable salt thereof

Furthermore, the present invention provides a health functional food composition for preventing or improving diseases caused by mitochondrial dysfunction, wherein the composition comprises the isoquinoline derivative compound or a pharmaceutically acceptable salt thereof as an active ingredient.

Additionally, the present invention provides a pharmaceutical composition for preventing or treating diseases caused by mitochondrial dysfunction, wherein the composition comprises the isoquinoline derivative compound or a pharmaceutically acceptable salt thereof as an active ingredient.

### Advantageous Effects

The pharmaceutical composition of the present invention for preventing or treating diseases caused by mitochondrial dysfunction, which comprises the isoquinoline derivative compound or a pharmaceutically acceptable salt thereof as an active ingredient, does not induce mitochondrial damage like conventional mitochondrial toxins such as CCCP, but specifically and excellently promotes the activity of mitophagy, thereby being able to improve mitochondrial abnormalities.

As such, it can be usefully employed in the treatment of diseases caused by mitochondrial dysfunction.

### BRIEF DESCRIPTION OF THE DRAWINGS

FIG. 1, comprising FIG. 1A through FIG. 1C, shows a result analyzing the effect of promoting mitophagy activity in normal human lung cell lines by the isoquinoline derivative compound of the present invention. FIG. 1A represents the analysis results using flow cytometry (FACS), FIG. 1B represents the analysis results using a confocal microscope, and FIG. 1C represents the measurement results of the quantitative changes in the mitochondria using a fluorescent protein, mito-YFP, which includes a targeting sequence to transport protein to the mitochondria.

FIG. 2, comprising FIG. 2A and FIG. 2B, shows a result analyzing the effect of promoting mitophagy activity in SH-SY5Y cell line (FIG. 2A) by the isoquinoline derivative compound of the present invention and a result analyzing the effect of promoting mitophagy activity in Hela-Parkin cell line (FIG. 2B) by the isoquinoline derivative compound of the present invention.

FIG. 3, comprising FIG. 3A and FIG. 3B, shows a result analyzing the effect of promoting mitophagy activity by the isoquinoline derivative compound of the present invention based on concentration (FIG. 3A) and time (FIG. 3B).

FIG. 4, comprising FIG. 4A and FIG. 4B, shows a result measuring the mitophagy activity (FIG. 4A) and autophagy activity (FIG. 4B) of the isoquinoline derivative compound of the present invention.

FIG. 5 shows a result analyzing the concentration-dependent effect of promoting mitophagy activity by the isoquinoline derivative compound of the present invention compared with palmatine and berberine.

FIG. 6 shows a result analyzing the levels of mitochondrial membrane potential and mitochondrial reactive oxygen species by the isoquinoline derivative compound of the present invention compared with CCCP.

FIG. 7 shows a result analyzing the mitophagy activity in the PINK knockdown cell line (shPINK1) by the isoquinoline derivative compound of the present invention compared with CCCP.

FIG. 8 shows a result measuring the ATP generation level in Alzheimer's disease cell model by the isoquinoline derivative compound of the present invention.

FIG. 9 and FIG. 10 show results from experiments on the therapeutic effects in an Alzheimer's disease animal model by the isoquinoline derivative compound of the present invention.

### BEST MODES OF THE INVENTION

Hereinafter, the present invention will be described in detail.

One embodiment of the present invention provides a pharmaceutical composition for preventing or treating diseases caused by mitochondrial dysfunction comprising as an active ingredient an isoquinoline derivative compound represented by the following Chemical Formula 1 or a pharmaceutically acceptable salt thereof:

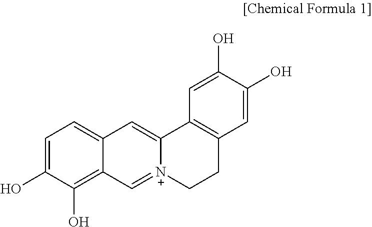 [SMILES: Oc1cc2c(cc1O)-c1cc3ccc(O)c(O)c3c[n+]1CC2]

In one embodiment of the present invention, the isoquinoline derivative compound may be produced by a method for preparing the isoquinoline derivative compound of claim 1 or a pharmaceutically acceptable salt thereof, comprising the step (step 1) of adding palmatine represented by Chemical Formula 2 or berberine represented by Chemical Formula 3 to an organic solvent and reacting with a Lewis acid catalyst, as in Reaction Formula 1 below, to produce the isoquinoline derivative compound represented Chemical Formula 1:

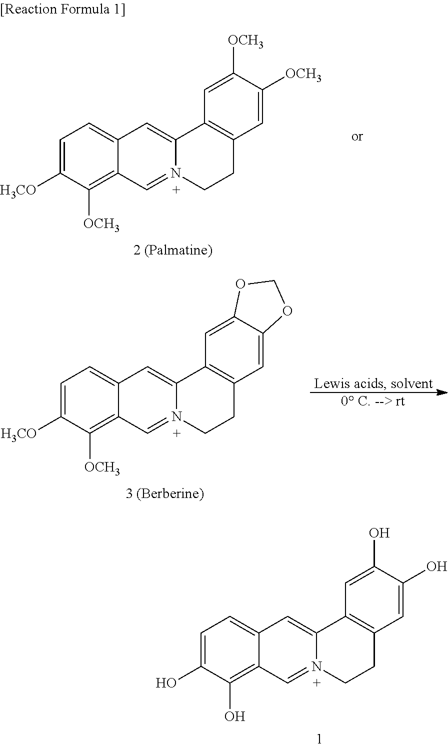

In one embodiment of the present invention, the Lewis acid catalyst may be at least one of metal halides such as BF₃, BBr₃, AlF₃, AlCl₃, AlBr₃, TiCl₄, TiBr₄, TiI₄, FeCl₃, FeCl₂, SnCl₂, SnCl₄, WCl₆, MoCl₅, SbCl₅, TeCl₂, ZnCl₂; metal alkyl compounds such as Et₃Al, Et₂AlCl, EtAlCl₂, Et₃Al₂Cl₃, (i-Bu)₃Al, (i-Bu)₂AlCl, (i-Bu)AlCl₂, Me₄Sn, Et₄Sn, Bu₄Sn, Bu₃SnCl; and metal alkoxy compounds such as Al(OR)₃₋ₓClₓ or Ti(OR)_{4-y}Cl_{y} (wherein the R represents an alkyl or aryl group, x is or 2, y is an integer of 1 to 3), for example, a metal halide, for example, BBr₃, but is not limited thereto.

In one embodiment of the present invention, the organic solvent may be any one and more selected from the group consisting of dimethyl sulfoxide, dimethyl formamide, acetone, tetrahydrofuran, benzene, toluene, ether, methanol, hexane, cyclohexane, pyridine, acetic acid, carbon tetrachloride, chloroform, dichloromethane, and water, for example, dichloromethane, but is not limited thereto.

In one embodiment of the present invention, the addition of the Lewis acid catalyst may be performed by using a method of adding to the palmatine dissolved in the organic solvent at about 0° C., in an inert gas atmosphere, for example, under a nitrogen stream.

In one embodiment of the present invention, after adding the Lewis acid catalyst, the reaction can be stirred for 10 to 14 hours, for example, 11 to 13 hours, for example, 12 hours, at room temperature, for example, 20° C. to 28° C., for example, 24° C. to 26° C., and the completion of the reaction may be confirmed, for example, using TLC (thin-layer Chromatography), but is not limited thereto.

In one embodiment of the present invention, the isoquinoline derivative compound or its pharmaceutically acceptable salt produced by the method of preparing the isoquinoline derivative compound may be a derivative in which a hydrophobic substituent (methoxy group) of the core structure of palmatine is substituted with a hydrophilic substituent or a functional group capable of providing intermolecular hydrogen bonding (hydroxy group), for example, may be one of 2,3,5,10-tetrahydroxy-5,6-dihydroisoquinolino[3,2-a]isoquinolin-7-ium bromide represented by the following Chemical Formula 1a, 2,3,9,10-tetrahydroxy-5,6-dihydroisoquinolino[3,2-a]isoquinolin-7-ium hydroxide represented by the following Chemical Formula 1b, and 2,3,9,10-tetrahydroxy-5,6-dihydroisoquinolino[3,2-a]isoquinolin-7-ium chloride represented by the following Chemical Formula 1c.

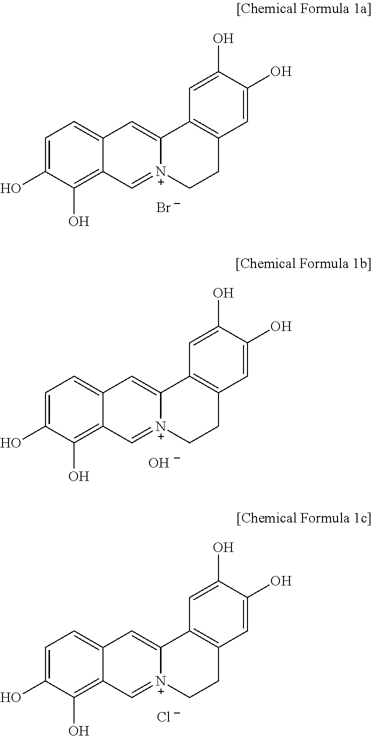

In one embodiment of the present invention, the isoquinoline derivative compound may promote the activity of mitophagy.

As used herein, the term “mitophagy” refers to the cellular degradation mechanism that removes damaged or unnecessary mitochondria, and when mitochondrial damage occurs, autophagosomes are formed, which can selectively degrade and remove the damaged mitochondria by fusion with lysosomes.

In one embodiment of the present invention, the disease caused by mitochondrial dysfunction may be any one and more selected from the group consisting of Alzheimer's disease, Huntington's Disease, amyotrophic lateral sclerosis (ALS), MELAS syndrome (mitochondrial encephalopathy, lactic acidosis, and stroke-like episodes), Charcot Marie Tooth disease (CMT), multiple sclerosis, Niemann-Pick disease, and dementia due to cerebral ischemia and cerebral hemorrhage. Preferably, it may be Alzheimer's disease, and more specifically, it may be Alzheimer's disease, but the present invention is not limited thereto.

### Pharmaceutically Acceptable Salt

The active ingredient of the present invention may be used in the form of a pharmaceutically acceptable salt, wherein salts formed by a pharmaceutically acceptable free acid are useful. The term “pharmaceutically acceptable salt” refers to any organic or inorganic additional salt of an active ingredient's basic compound, wherein the salt, at concentrations demonstrating relatively non-toxic and harmless effective actions in patients, does not reduce the beneficial effects of the basic compound of the active ingredient due to side effects attributed to the salt. These salts may use inorganic acids or organic acids as free acids. For inorganic acids, hydrochloric acid, bromic acid, nitric acid, sulfuric acid, hypochlorous acid, phosphoric acid, and the like may be used. For organic acids, citric acid, acetic acid, actic acid, maleic acid, fumaric acid, gluconic acid, methanesulfonic acid, glyconic acid, succinic acid, tartaric acid, galacturonic acid, embonic acid, glutamic acid, aspartic acid, oxalic acid, (D) or (L) malic acid, ethanesulfonic acid, 4-toluenesulfonic acid, salicylic acid, citric acid, benzoic acid, or malonic acid and the like may be used. Also, these salts comprise alkali metal salts (such as sodium salt, potassium salt) and alkali earth metal salts (such as calcium salt, magnesium salt). For example, the acid addition salts may comprise acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hybenzoate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate, aluminum, arginine, benzathine, calcium, choline, diethylamine, diolamine, glycine, lysine, magnesium, meglumine, olamine, potassium, sodium, tromethamine, zinc salt and the like, among which hydrochloride or trifluoroacetate is preferable.

According to the present invention, the acid addition salt may be prepared by a conventional method, for example, by dissolving the active ingredient in an organic solvent, for example, methanol, ethanol, acetone, methylene chloride, acetonitrile and the like, adding organic or inorganic acid to produce a precipitate, filtering, and drying the precipitate, or by distilling under reduced pressure after adding solvent and an excess of acid, and then drying it, or crystallizing in an organic solvent.

Also, a pharmaceutically acceptable metal salt may be made using a base. Alkali metal or alkali earth metal salts, for example, may be made by dissolving the compound in an excess of alkali metal hydroxide or alkali earth metal hydroxide solution, filtering an insoluble compound salt, and then evaporating and drying the filtrate. In this case, it is pharmaceutically appropriate to produce sodium, potassium, or calcium salts. Moreover, the corresponding silver salt may be obtained by reacting an alkali metal or alkali earth metal salt with an appropriate silver salt (e.g., silver nitrate).

Furthermore, the present invention comprises not only the active ingredient and its pharmaceutically acceptable salt, but also all possible solvates, hydrates, isomers, optical isomers, and the like that may be manufactured therefrom.

### Pharmaceutical Composition

The active ingredient of the present invention may be administered in various forms, both orally and non-orally, when administered clinically. When formulated, it is typically prepared using commonly used diluents or excipients such as fillers, bulking agents, binders, wetting agents, disintegrants, surfactants, and the like.

For oral administration, solid formulations comprise tablets, pills, powders, granules, capsules, troches, and the like. Such solid formulations comprise at least one active ingredient of the present invention and at least one excipient, for example, starch, calcium carbonate, sucrose, lactose, or gelatin, mixed together. Furthermore, lubricants such as magnesium stearate and talc are also used. Liquid formulations for oral administration comprise suspensions, solutions, emulsions, or syrups, which may comprise commonly used simple diluents like water and liquid paraffin as well as various excipients, for example, wetting agents, sweeteners, fragrances, and preservatives.

Formulations for non-oral administration comprise sterile aqueous solutions, non-aqueous solvents, suspending agents, oils, freeze-dried preparations, and suppositories. Non-aqueous solvents and suspending agents may comprise propylene glycol, polyethylene glycol, vegetable oils like olive oil, injectable esters like ethyl oleate, and the like. For the base of suppositories, materials such as witepsol, macrogol, tween 61, cocoa butter, laurin butter, glycerol, and gelatin may be used.

Additionally, the effective dose of the active material of the present invention for humans may vary depending on the patient's age, weight, gender, form of administration, health status, and the severity of the disease, and is generally about 0.001-100 mg/kg/day, preferably 0.01-35 mg/kg/day. Based on an adult patient weighing 70 kg, it is generally 0.07-7000 mg/day, and preferably 0.7-2500 mg/day. The dose may be divided and administered once or multiple times a day, depending on the judgment of a physician or pharmacist.

### Health Food and Health Functional Food Composition

There is no particular limitation to the types of foods. Examples of foods to which the active material of the present invention may be added comprise drinks, meats, sausages, breads, biscuits, rice cakes, chocolates, candies, snacks, confections, pizzas, ramen, other noodles, gums, dairy products including ice creams, various soups, beverages, alcoholic drinks, vitamin complexes, dairy products, and processed dairy products, and comprise both conventional health foods and health functional food compositions.

A health food and health functional food composition comprising the active material according to the present invention may be directly added to food or used together with other foods or food ingredients and may be used appropriately according to conventional methods. The mixture amount of the active material may be suitably determined according to its intended purpose (for preventing or improving). Generally, the amount of the composition in health foods and health functional foods may be added in the range of 0.1 to 90 weight parts based on the total weight of the food. However, for long-term consumption aimed at maintaining health or regulating health, the amount may be below the stated range, and as there is no safety issue, the active ingredient may be used in amounts above the range.

The health food and health functional food composition of the present invention, besides comprising the active material of the present invention as an essential ingredient in the indicated proportions, may comprise various flavoring agents or natural carbohydrates as additional ingredients without any particular limitation on other ingredients. Examples of the natural carbohydrates comprise common sugars such as monosaccharides, e.g., glucose, fructose; disaccharides, e.g., maltose, sucrose; and polysaccharides, e.g., dextrin, cyclodextrin; and sugar alcohols such as xylitol, sorbitol, and erythritol. Other flavoring agents may beneficially comprise natural flavorings like thaumatin, stevia extracts (e.g., rebaudioside A, glycyrrhizin) and synthetic flavorings like saccharin, aspartame. The proportion of the natural carbohydrates is generally about 1 to 20 g per 100 of the health functional food composition of the present invention, and preferably about 5 to 12 g.

Additionally, the health food and health functional food composition comprising the active material of the present invention may comprise various nutrients, vitamins, minerals (electrolytes), synthetic flavorings and natural flavorings, colorants and enhancers (cheese, chocolate, etc.), pectic acid and its salts, alginic acid and its salts, organic acids, protective colloid thickeners, pH adjusters, stabilizers, preservatives, glycerin, alcohol, carbonators for carbonated drinks, and the like. Furthermore, the health food and health functional food composition of the present invention may comprise fruit pulp for the production of natural fruit juices, fruit juice beverages, and vegetable drinks.

Such ingredients may be used independently or in combination. The proportion of such additives is not particularly critical but is typically selected in the range of 0.1 to about 20 weight parts per 100 weight parts of the health food and health functional food composition comprising the active material of the present invention.

The present invention will be described in more detail by examples. These examples are merely for the purpose of illustrating the present invention in more detail and it should be evident to those with ordinary skill in the art that the scope of the present invention is not limited thereto.

### EXAMPLES

### Example 1. Synthesis of Isoquinoline Derivative Compound—Synthesis of 2,3,5,10-Tetrahydroxy-5,6-dihydroisoquinolino[3,2-a]isoquinolin-7-iumbromide (CD1-012)

In a dried 250 mL round flask, palmatine (1.0 g, 2.92 mmol) was dissolved in 40 mL of anhydrous CH₂Cl₂. A BBr₃ solution (12.80 mL, 12.80 mmol) was added under a nitrogen atmosphere at 0° C. to prepare the reaction mixture. The reaction mixture was stirred at room temperature for 12 hours, and the completion of the reaction was confirmed using TLC. After the completion of the reaction, 10 mL of MeOH was added to the reaction mixture and stirred for 30 minutes. It was then concentrated via a vacuum concentrator, washed with CH₂Cl₂ (100 mL×5), and dried under reduced pressure to yield a solid form of the isoquinoline derivative compound (2,3,9,10-Tetrahydroxy-5,6-dihydroisoquinolino[3,2-a]isoquinolin-7-iumbromide; CD1-012) with a yield of 99% (1.09 g, 2.98 mmol). The reaction is represented by Reaction Formula 1a below:

¹-NMR (400 MHz, DMSO-d₆) δ10.71 (s, 1H), 10.62 (s, 1H) 9.99 (br, 1H), 9.74 (s, 1H), 9.23 (br, 1H), 8.58 (s, 1H), 7.75 (d, J=8.8 Hz, 1H) 7.61 (d, J=8.8 Hz 1H), 7.46 (s, 1H), 6.77 (s, 1H), 4.82 (t, J=6.0 Hz, 2H), 3.06 (t, J=6.0 Hz, 2H)

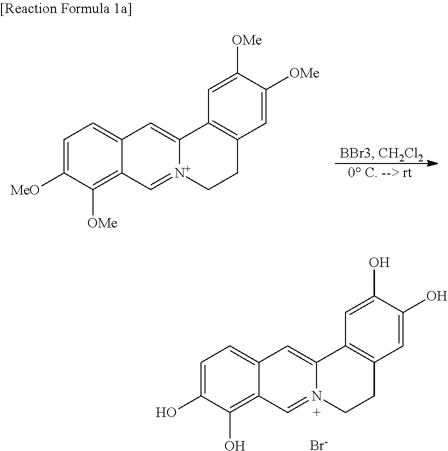

### Comparative Example 1. Carbonyl Cyanide 3-chlorophenylhydrazone (CCCP)

CCCP, a representative compound promoting mitophagy, was used as Comparative Example 1.

### Comparative Example 2. Palmatine

Palmatine (CAS Number: 486-67-7), represented by Chemical Formula 2 below, was used as Comparative Example 2.

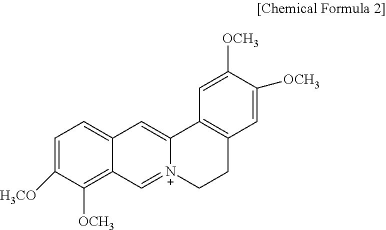 [SMILES: COc1cc2c(cc1OC)-c1cc3ccc(OC)c(OC)c3c[n+]1CC2]

### Comparative Example 3. Berberine

Berberine, represented by Chemical Formula 3 below and with CAS Numbers: 633-65-8(Berberine·HCl) or 2086-83-1(Berberine·HCl·2H₂O), was used as Comparative Example 3.

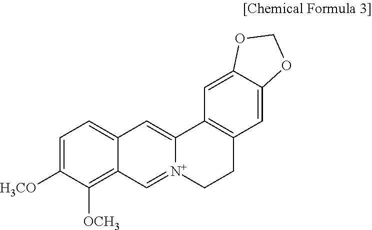 [SMILES: COc1ccc2cc3[n+](cc2c1OC)CCc1cc2c(cc1-3)OCO2]

### Experimental Example 1. Analysis of Mitophagy Activity Promoting Effect

To analyze the mitophagy-promoting activity effect of the isoquinoline derivative compound synthesized in the Example 1 (hereinafter, CD1-012), a quantification method for mitophagy activity, using a mt-Keima fluorescent protein, was utilized, which can measure the mitophagy activity in living cells through flow cytometry or a confocal microscope.

### Experimental Example 1-1. Analysis of Mitophagy Activity Promoting Effect

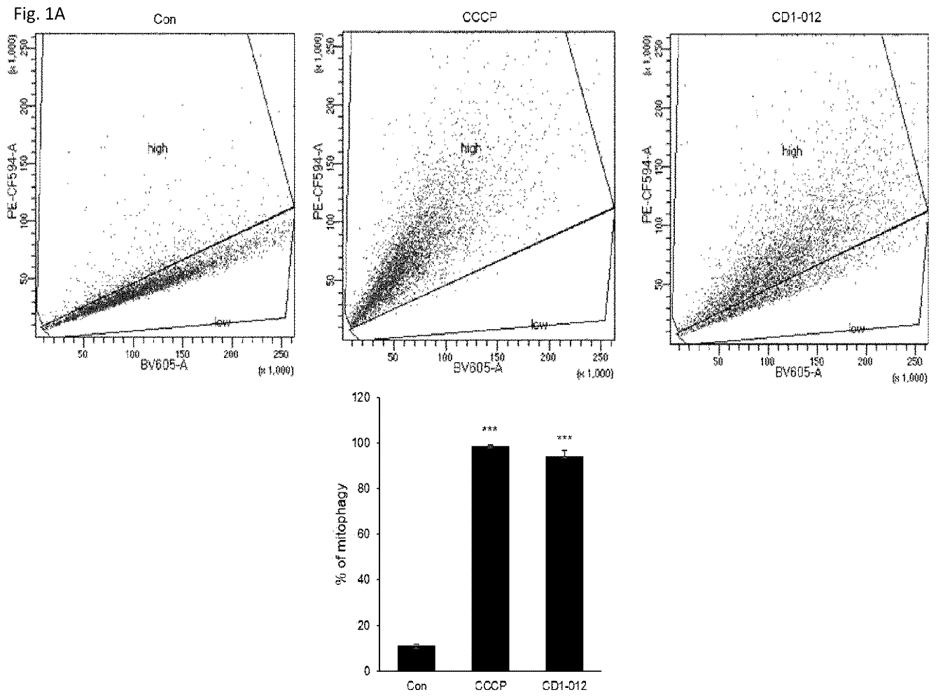

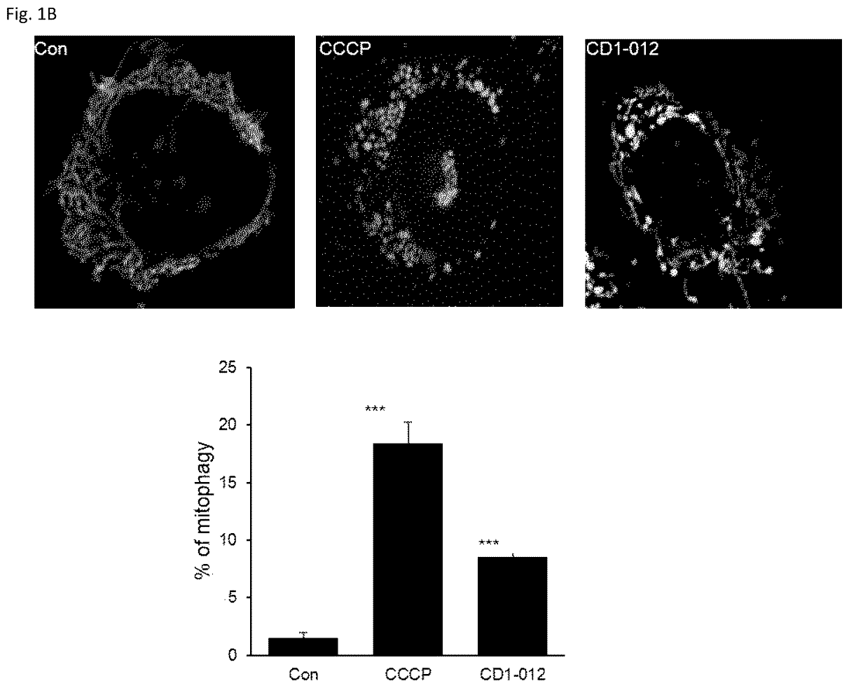

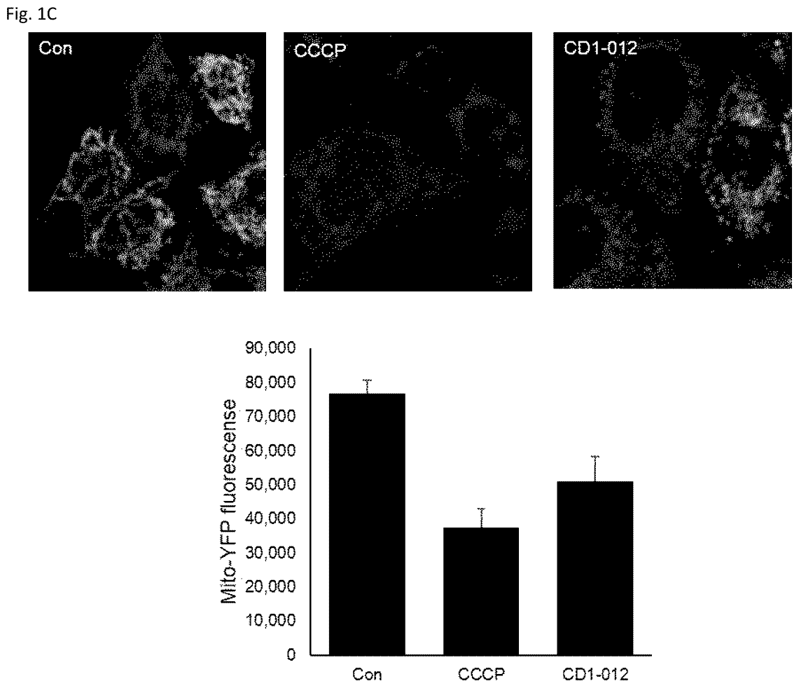

To analyze the mitophagy-promoting activity effect of CD1-012 synthesized in Example 1, the BEAS-2B cell line, which is a human normal lung cell line, was induced to express the mt-Keima fluorescent protein. Following this, the CD1-012 (15 μM) and the compound of Comparative Example 1, CCCP (10 μM), were treated for 24 hours. After the treatment, the mitophagy activity of each sample was measured, and the results were represented in FIG. 1.

Specifically, FIG. 1A represents the analysis results using flow cytometry (FACS), FIG. 1B represents the analysis results using a confocal microscope, and FIG. 1C represents the measurement results of the quantitative changes in the mitochondria using a fluorescent protein, mito-YFP, which includes a targeting sequence to transport protein to the mitochondria.

Referring to FIG. 1, it was observed that the samples treated with CD1-012 showed a significant increase in mitophagy activity compared to the untreated control group (Con). Especially in FIG. 1A, it was observed that the mitophagy activity of the sample treated with CD-012 was increased as significantly as that of the sample treated with the representative mitophagy promoting compound, CCCP.

### Experimental Example 1-2. Analysis of Mitophagy Activity Promoting Effect in Various Cell Lines

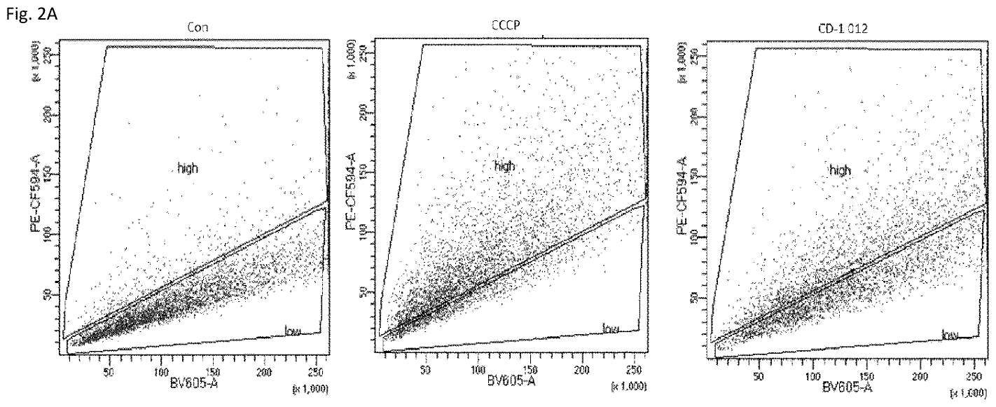

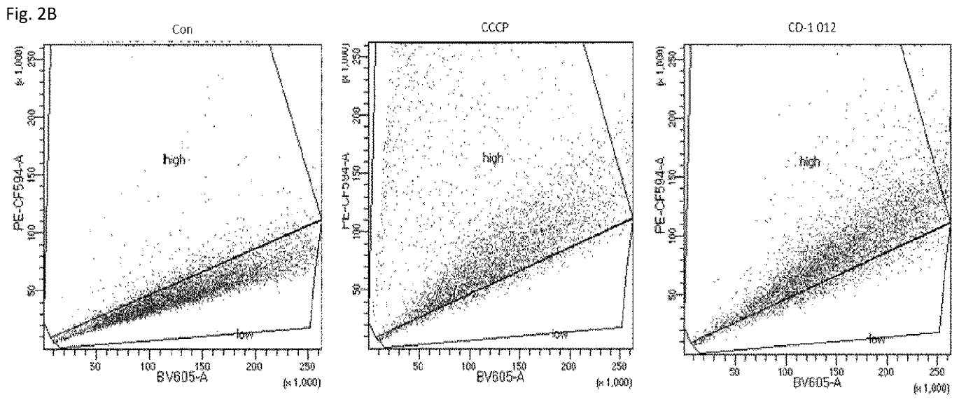

To determine whether CD1-012 synthesized in Example 1 enhances mitophagy activity in various cell lines, the human neuroblastoma cell line, SH-SY5Y, expressing mt-Keima fluorescent protein and HeLa cervical cancer cell line (Hela-Parkin) expressing Parkin (E3ligase) were treated with CD1-012 and CCCP of Comparative Example 1. Following this treatment, the mitophagy activity of each sample was analyzed using flow cytometry (FACS), and the results were represented in FIG. 2.

Specifically, FIG. 2A represents the analysis results for the SH-SY5Y cell line, and FIG. 2B represents the analysis results for the HeLa-Parkin cell line.

Referring to FIG. 2, when treating with CD1-012 of Example 1, a significant increase in mitophagy activity was observed compared to the control group (Con). This confirmed the mitophagy activity promoting effect in various cell lines.

### Experimental Example 1-3. Analysis of Mitophagy Activity Promoting Effect Depending on Concentration and Time

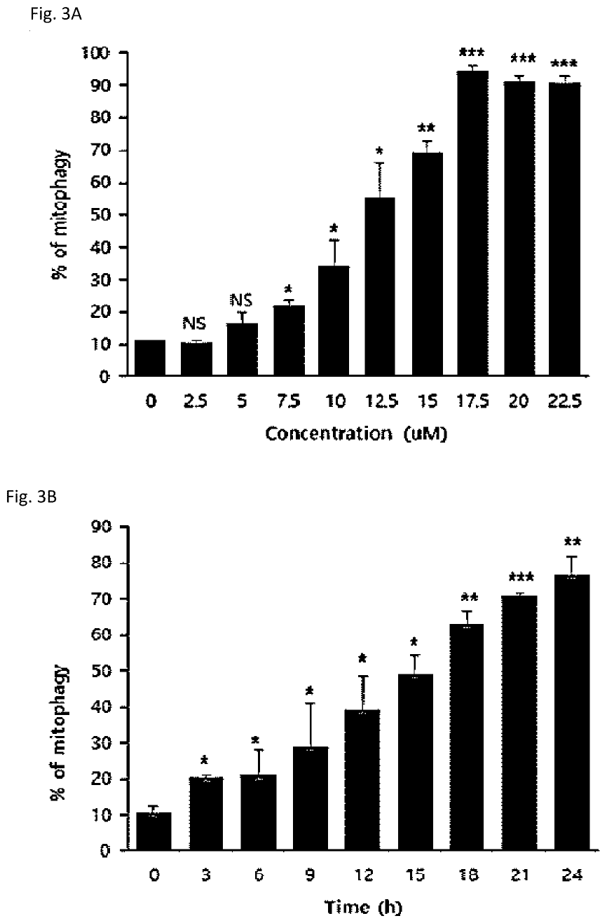

To analyze whether CD1-012 synthesized in Example 1 promotes mitophagy activity in a concentration and time-dependent manner, the BEAS-2B cell line expressing mt-Keima was treated with varying concentrations of CD1-012 or a constant concentration (15 μM) for different durations. The mitophagy activity was then measured using flow cytometry, with the concentration-dependent mitophagy activity results represented in FIG. 3A and the time-dependent mitophagy activity results in FIG. 3B.

Referring to FIG. 3A, it was observed that starting from a concentration of 7.5 μM of CD1-012, the activity began to significantly increase and continued to do so in a concentration-dependent manner up to 17.5 μM. Referring to FIG. 3B, after treating with CD1-012, a significant increase in mitophagy activity was observed starting from 3 hours, reaching its maximum at hours.

Such a pattern of increase in mitophagy suggests that CD1-012 directly increases mitophagy activity in a concentration and time-dependent manner.

### Experimental Example 1-4. Confirmation of Specific Mitophagy Induction

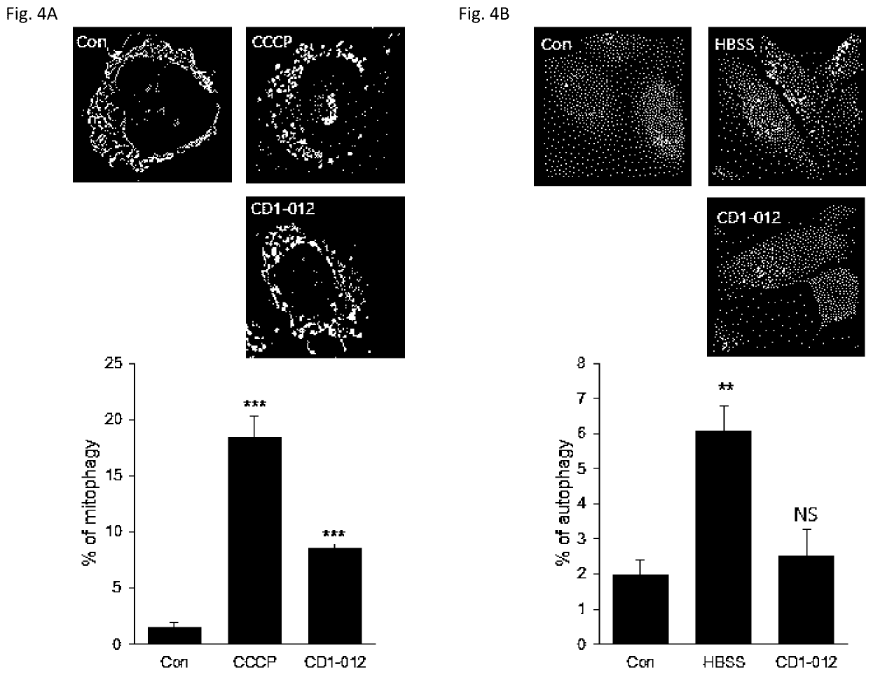

To confirm whether the synthesized CD1-012 in Example 1 specifically increases mitophagy activity, the CD1-012 (15 μM) of the Example 1 and the CCCP (10 μM) of Comparative Example 1 were treated to the BEAS-2B cell line expressing mt-Keima for 18 hours. Subsequently, the mitophagy activity was analyzed using a confocal microscope, and the results were represented in FIG. 4A. Moreover, the BEAS-2B cell line expressing Keima fluorescent protein was cultured in HBSS (Hanks' balanced salts solution) for 3 hours to induce a nutrient-deficient state (starvation, starv.). The autophagy activity was measured by comparing the sample treated with the CD1-012 (15 μM) for hours using a confocal microscope, and the results were represented in FIG. 4B.

Referring to FIG. 4A, it was observed that the sample treated with CD1-012 had an effect of promoting mitophagy activity similar to the sample treated with CCCP. Referring to FIG. 4B, although the sample induced with nutrient deficiency (HBSS) induced autophagy, the sample treated with CD1-012 did not increase autophagy activity.

It was confirmed that CD1-012 is a compound that specifically increases only the activity of mitophagy.

### Experimental Example 1-5. Comparison of Mitophagy Activity Induction Effects with Berberine and Palmitate

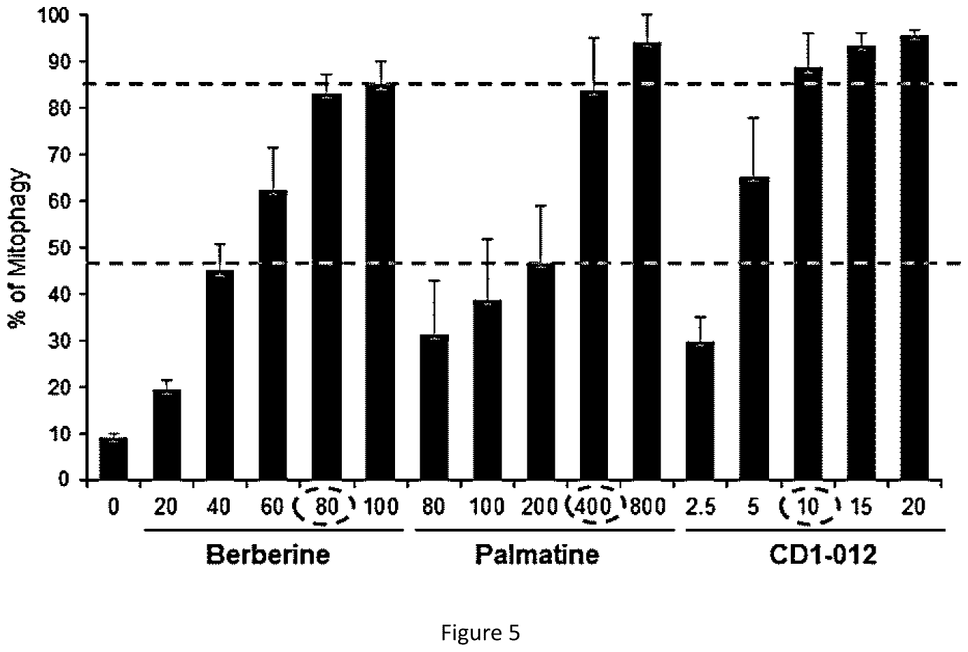

To verify that the CD1-012 synthesized in Example 1 improved mitophagy activity compared to palmitate and berberine, the CD1-012, palmitate of Comparative Example 2, and berberine of Comparative Example 3 were treated at various concentrations to the human normal lung cell line BEAS-2B expressing mt-Keima fluorescent protein. The mitophagy-promoting activity effect of each sample was compared, and the results were represented in FIG. 5.

Referring to FIG. 5, while palmitate reached maximum mitophagy activity at 400 μM and berberine at 80 μM, CD1-012 exhibited similar mitophagy activity at 10 μM. It was confirmed that the mitophagy-promoting activity effect of CD1-012 was approximately 8 times better than the berberine and about 40 times better than the palmitate.

### Experimental Example 2. Analysis of Induction of Mitochondrial Dysfunction

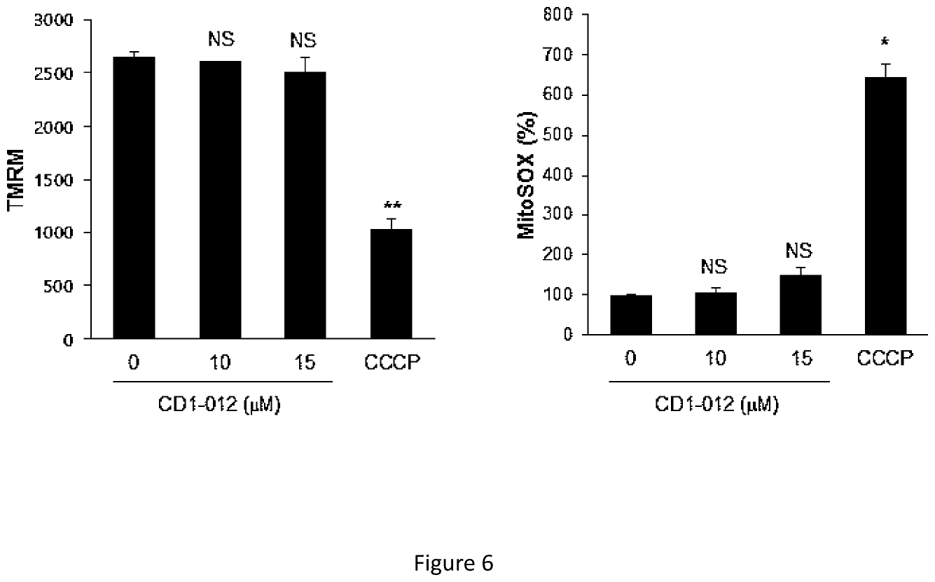

To determine if the CD1-012 synthesized in Example 1 induces mitochondrial dysfunction similar to CCCP of Comparative Example 1, the CD1-012 (10 μM or 15 μM) and the CCCP (10 μM) were treated for hours. Afterward, the mitochondrial membrane potential and the level of mitochondrial reactive oxygen species (ROS) of each sample were analyzed, and the results were represented in FIG. 6.

The mitochondrial membrane potential was analyzed using the TMRM (tetramethylrhodamine methyl ester) assay, and the mitochondrial ROS was analyzed using the MitoSOX assay.

Referring to FIG. 6, while the sample treated with CCCP significantly reduced the mitochondrial membrane potential, the sample treated with CD1-012 did not observe a decrease in the mitochondrial membrane potential. Also, while the sample treated with CCCP significantly increased mitochondrial ROS, the sample treated with CD1-012 did not increase mitochondrial ROS.

It was confirmed that unlike CCCP, which reduces the mitochondrial membrane potential and induces mitochondrial dysfunction, thus increasing mitophagy activity, CD1-012 is a compound that does not induce mitochondrial dysfunction.

### Experimental Example 3 . Confirmation of PINK1-Parkin Pathway-Independent Mitophagy Activation

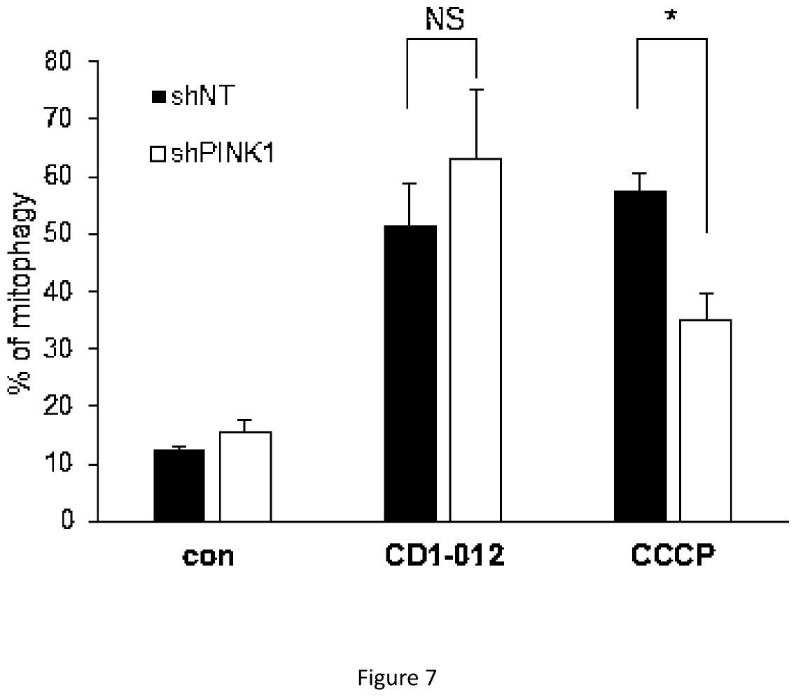

In order to analyze whether the activation of mitophagy by the CD1-012 synthesized in Example 1 is dependent on the PINK1-Parkin pathway, the PINK 1 was knockdown using short hairpin RNA (shRNA) in BEAS-2B cell lines. Then, CCCP (10 μM) from Comparative Example 1 and the CD1-012 (15 μM) were treated for 18 hours, and the mitophagy activity of each sample was analyzed using a flow cytometry (FACS) and represented in FIG. 7.

Referring to FIG. 7, mitophagy activation by CCCP in PINK1 knockdown cells (shPINK1) significantly decreased compared to the control cell line (shNT), but the mitophagy activation by CD1-012 did not show any significant difference.

Through these results, it was confirmed that the activation of mitophagy by CD1-012 activates mitophagy independently of the stress-mediated PINK1-Parkin pathway.

### Experimental Example 4. Confirmation of the Mitochondrial Function Improvement Effect in Alzheimer's Disease Cell Model

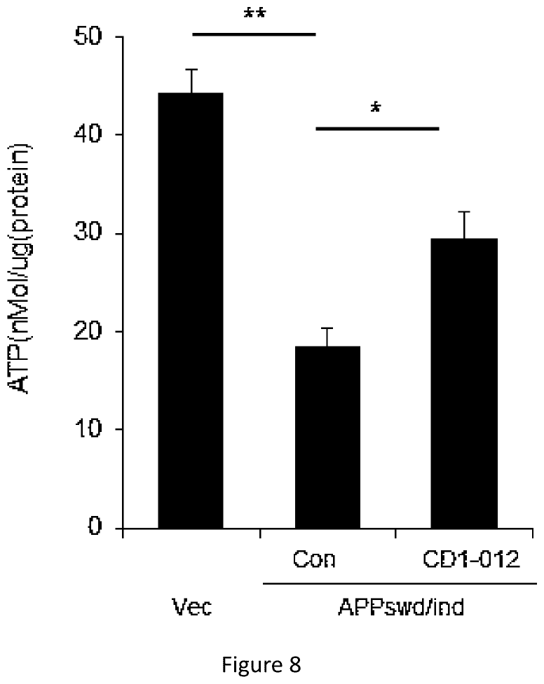

To verify whether the CD1-012 synthesized in Example 1 improves mitochondrial dysfunction in the Alzheimer's disease cell model, the human neuron cell line SH-SY5Y was overexpressed with APPswd/ind protein causing Alzheimer's disease to prepare the Alzheimer's disease cell model. After treating the cell model with the CD1-012 (20 μM) for hours, the ATP generation level, an indicator of mitochondrial function, was measured 48 hours later and the results were represented in FIG. 8.

Referring to FIG. 8, in the cells where the APPswd/ind protein was overexpressed, the ATP production level decreased compared to normal cells. After treating with the CD1-012, the ATP production level in the Alzheimer's disease cells was observed to recover and increase. Through these results, it was confirmed that CD1-012 may improve mitochondrial dysfunction in the Alzheimer's disease cell model.

### Experimental Example 5. Verification of the Effect in Animal Models

### Experimental Example 5-1. Therapeutic Effect in Alzheimer's Disease Animal Model

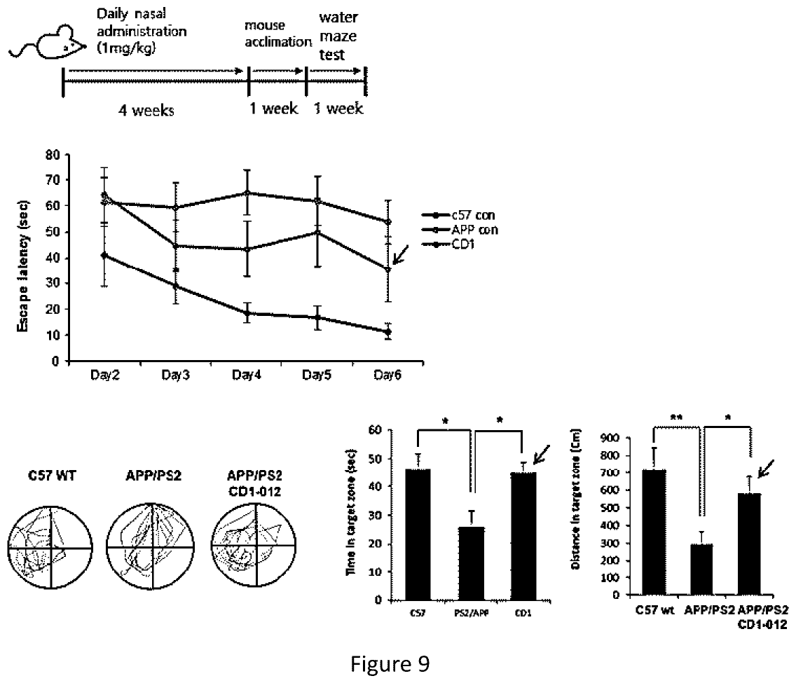

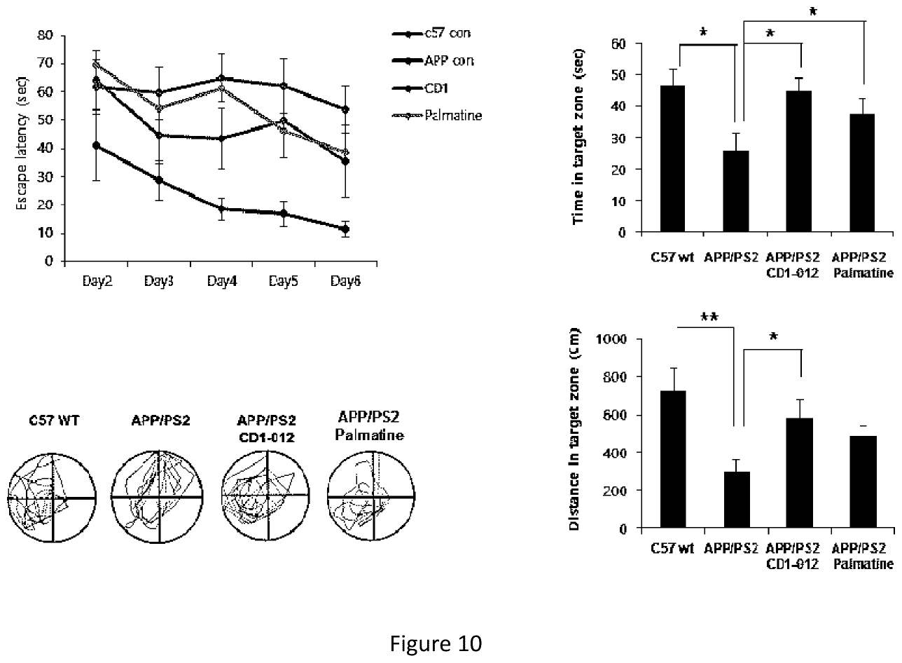

To confirm whether the CD1-012 synthesized in Example 1 exhibits a therapeutic effect in the Alzheimer's disease animal model, the Alzheimer's disease mouse model C7-Tg(NSE-hPS2*N1411):Tg(NSE-hAPPsw)/Korl(APP/PS2) was treated with the CD1-012 at a concentration of 1 mg/kg every day for 4 weeks via nasal administration. Subsequently, to analyze the representative symptom improvement effect of Alzheimer's disease, spatial learning and memory abilities were measured through the Morris water maze test (represented in FIG. 10, 9-10 mice per group). During the behavior test, from day 1 to day 6, training was conducted, where the time taken from the start to climb the escape stand was measured 4 times. On the 7th day, free-swimming test was conducted for 60 seconds, and the time spent in the area where the escape stand was located during training was measured to analyze the learning and memory abilities of the experimental animals. The results were represented in FIG. 9.

Referring to FIG. 9, normal control mice showed a learning effect, reducing the time to find the escape platform (escape latency) over 6 days of training. In contrast, the Alzheimer's disease model APP/PS2 mice did not show a learning effect over 6 days, and the free-swimming analysis on the 7 th day also showed reduced time and distance spent in the escape platform area, confirming memory impairment.

On the other hand, APP/PS2 mice treated with CD-012 showed a learning effect from day 3 to day 6, and on the 7th day of the free-swimming analysis, the time spent near the escape platform recovered close to that of normal mice. Through these results, it was confirmed that CD1-012 significantly improved the learning and memory ability of Alzheimer's disease animals.

### Experimental Example 5-2. Comparative Treatment Effects of Palmatine and Alzheimer's Disease Animal Model

For the purpose of comparing the treatment effect of the CD1-012 synthesized in Example 1 with that of palmatine in Comparative Example 2 on an Alzheimer's disease animal model, APP/PS2 mice were intranasally administered with the CD1-012 at a concentration of 1 mg/Kg and palmatine at 10 mg/Kg every day for 4 weeks. The treatment effects on dementia were confirmed with a water maze test, and the results were represented in FIG. 10 (9-10 mice per group).

Referring to FIG. 10, it was observed that both the CD1-012 treated group and the palmatine treated group had similar extents of learning effects during 6 days of training, and on the 7th day in the free-swimming analysis, it was confirmed that the memory capabilities of both groups improved similarly. Through these results, it was confirmed that CD1-012 has a similar dementia treatment effect at a concentration 10 times lower than that of palmatine.

From the results of the above-mentioned examples and experimental examples, the isoquinoline derivative compound of the present invention was found to induce mitophagy and remove dysfunctional mitochondria. Specifically, it was found to increase mitophagy activity specifically, without inducing damage to the mitochondria, activate mitophagy specifically, and activate mitophagy in a manner independent of the PINK1-Parkin pathway mediating stress-induced mitophagy. It was further observed that the compound improves learning effects and memory capabilities in animal models of diseases caused by mitochondrial dysfunction, specifically, in Alzheimer's disease, thereby effectively improving cognitive function and confirming that it can treat diseases caused by mitochondrial dysfunction.

### Manufacturing Example of Drug

The active material according to the present invention may be formulated in various forms depending on its purpose. The following are some methods of formulating the active material according to the present invention comprising the active ingredient, and the present invention is not limited thereto.

### <Manufacturing Example of Drug 1> Manufacturing of Acid Formulation

Active material 2 g

Lactose 1 g

The ingredients were mixed and filled in a sealed pack to prepare the acid formulation.

### <Manufacturing Example of Drug 2> Manufacturing of Tablet

Active material 100 mg

Cornstarch 100 mg

Lactose 100 mg

Magnesium stearate 2 mg

The ingredients were mixed and then tableted according to conventional tablet manufacturing methods to prepare the tablet.

### <Manufacturing Example of Drug 3> Manufacturing of Capsule

Active material 100 mg

Cornstarch 100 mg

Lactose 100 mg

Magnesium stearate 2 mg

The ingredients were mixed and then filled into gelatin capsules according to conventional capsule manufacturing methods to prepare the capsule formulation.

### <Manufacturing Example of Drug 4> Manufacturing of Injectable

Active material 10 μg/mL

Diluted hydrochloric acid BP until pH 3.5

Injectable sodium chloride BP up to 1 mL

The active material according to the present invention was dissolved in an appropriate volume of injectable sodium chloride BP, and the pH of the resulting solution was adjusted to pH 3.5 using diluted hydrochloric acid BP. The volume was adjusted using injectable sodium chloride BP and mixed thoroughly. The solution was filled into transparent glass 5 mL type I ampoules, sealed under an air upper grid by melting the glass, and sterilized by autoclaving at 120° C. for at least 15 minutes to prepare the injectable.

### <Manufacturing Example of Drug 5> Manufacturing of Nasal Spray

Active material 1.0 g

Sodium acetate 0.3 g

Methylparaben 0.1 g

Propylparaben 0.02 g

Sodium chloride appropriate amount

HCl or NaOH for pH adjustment appropriate amount

Purified water appropriate amount

According to conventional nasal spray manufacturing methods, it was prepared to contain 3 mg of active material per 1 mL of saline (0.9% NaCl, w/v, solvent is purified water). It was filled into an opaque spray container and sterilized to prepare the nasal spray.

### <Manufacturing Example of Drug 6> Manufacturing of Liquid Formulation

Active material 100 mg

High-fructose corn syrup 10 g

Mannitol 5 g

Purified water appropriate amount

Each ingredient was dissolved in purified water according to conventional liquid formulation manufacturing methods, lemon flavor was added, and then the ingredients were mixed. Purified water was added to adjust the total to 100 mL, and it was filled into a brown bottle and sterilized to prepare the liquid formulation.

### Manufacturing Example of Health Food

The active material according to the present invention may be manufactured into various forms of health food depending on its purpose. The following are examples of manufacturing health foods comprising an active material as an active ingredient according to the present invention, but the present invention is not limited thereto.

### <Manufacturing Example of Health Food 1> Manufacturing of Dairy Products

0.01-1 weight part of the active material of the present invention was added to milk, and various dairy products such as butter and ice cream were manufactured using the milk.

### <Manufacturing Example of Health Food 2> Manufacturing of Seonsik

Brown rice, barley, glutinous rice, and barnyard millet were gelatinized by a known method and dried, and then mixed and ground into a powder of 60 mesh. Black beans, black sesame, and perilla seeds were also steamed by a known method, dried, and then mixed and ground into a powder of 60 mesh. The active material of the present invention was concentrated under reduced pressure in a vacuum concentrator, and a dried powder was obtained. The following proportions of the manufactured cereals, seeds, and dried powder of the active material were mixed.

Cereals (brown rice 34 weight parts, barnyard millet 19 weight parts, barley 20 weight parts),

Seeds (perilla seeds 7 weight parts, black beans 8 weight parts, black sesame 7 weight parts),

Active material (2 weight parts),

Ganoderma lucidum (1.5 weight parts), and

Rehmannia glutinosa (1.5 weight parts).

### Manufacturing Example of Health Functional Food Composition

Active materials according to the present invention may be manufactured into various forms of health functional food compositions depending on its purpose. The following are examples of manufacturing health functional food compositions comprising an active material as an active ingredient according to the present invention, but the present invention is not limited thereto.

### <Manufacturing Example of Health Functional Food Composition 1> Manufacturing of Health Functional Food Composition

Active material 100 mg

Vitamin mixture appropriate amount

Vitamin A acetate 70 μg

Vitamin E 1.0 mg

Vitamin B1 0.13 mg

Vitamin B2 0.15 mg

Vitamin B6 0.5 mg

Vitamin B12 0.2 μg

Vitamin C 10 mg

Biotin 10 μg

Nicotinamide 1.7 mg

Folic acid 50 μg

Pantothenic acid calcium 0.5 mg

Mineral mixture appropriate amount

Ferrous sulfate 1.75 mg

Zinc oxide 0.82 mg

Magnesium carbonate 25.3 mg

Potassium dihydrogen phosphate 15 mg

Calcium hydrogen phosphate 55 mg

Potassium citrate 90 mg

Calcium carbonate 100 mg

Magnesium chloride 24.8 mg

The composition ratio of the vitamins and minerals mixture was mixed as a desirable example suitable for relatively health functional foods, but it is acceptable to modify the mixing ratio at will. The above ingredients were mixed and then granulated, and may be used to manufacture the health functional food composition according to a conventional method.

### <Manufacturing Example of Health Functional Food Composition 2> Manufacturing of Health Functional Beverage

Active material 100 mg

Citric acid 100 mg

Oligosaccharide 100 mg

Plum concentrate 2 mg

Taurine 100 mg

Add purified water to make a total of 500 mL

The above ingredients were mixed and then heated with stirring at 85° C. for about 1 hour. The resulting solution was filtered, filled into a sterilized container, and sterilized by sealing. After refrigeration, it may be used to manufacture the health beverage composition of the present invention. The composition ratio was mixed as a desirable example suitable for relatively preferred beverages, but it may be modified according to regional and ethnic preferences, demand groups, demand countries, and usage purposes.

The preferable examples of the present invention have been described so far. Those with ordinary knowledge in the technical field to which the present invention pertains will understand that the present invention can be embodied in modified forms without departing from the essential characteristics of the present invention. Therefore, the described examples should be considered in a descriptive perspective, not a limiting one. The scope of the present invention is not limited to the aforementioned description but is particularly represented in the claims, and all variations within the equivalent scope should be interpreted as being included in the present invention.

1-14. (canceled)

15. A method for treating a disease caused by mitochondrial dysfunction, comprising the step of administering a composition comprising an isoquinoline derivative compound represented by Chemical Formula 1 below, or a pharmaceutically acceptable salt thereof as an active ingredient to a subject in need thereof:

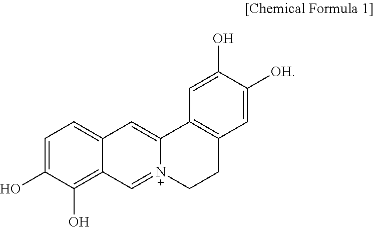 [SMILES: Oc1cc2c(cc1O)-c1cc3ccc(O)c(O)c3c[n+]1CC2]

16. The method of claim 15, wherein the isoquinoline derivative compound or a pharmaceutically acceptable salt thereof promotes the activity of mitophagy.

17. The method of claim 16, wherein the activity of mitophagy is independent of the PINK1-Parkin pathway.

18. The method of claim 15, wherein the disease caused by mitochondrial dysfunction is any one or more selected from the group consisting of Alzheimer's disease, Huntington's Disease, amyotrophic lateral sclerosis (ALS), MELAS syndrome (mitochondrial encephalopathy, lactic acidosis, and stroke-like episodes), Charcot Marie Tooth disease (CMT), multiple sclerosis, Niemann-Pick disease, and dementia due to cerebral ischemia and cerebral hemorrhage.

19. The method of claim 15, wherein the isoquinoline derivative compound or a pharmaceutically acceptable salt thereof may be characterized by any one or more of the following:
a) specifically increasing mitophagy activity, not autophagy activity; and
b) not inducing mitochondrial dysfunction.

20. A composition for improving or treating a disease caused by mitochondrial dysfunction, comprising as an active ingredient an isoquinoline derivative compound represented by Chemical Formula 1 below or a pharmaceutically acceptable salt thereof:

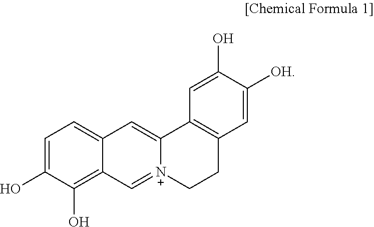 [SMILES: Oc1cc2c(cc1O)-c1cc3ccc(O)c(O)c3c[n+]1CC2]

21. The composition of claim 20, wherein the isoquinoline derivative compound or a pharmaceutically acceptable salt thereof promotes the activity of mitophagy.

22. The composition of claim 20, wherein the activity of mitophagy is independent of the PINK1-Parkin pathway.

23. The composition of claim 20, wherein the disease caused by mitochondrial dysfunction is any one or more selected from the group consisting of Alzheimer's disease, Huntington's Disease, amyotrophic lateral sclerosis (ALS), MELAS syndrome (mitochondrial encephalopathy, lactic acidosis, and stroke-like episodes), Charcot Marie Tooth disease (CMT), multiple sclerosis, Niemann-Pick disease, and dementia due to cerebral ischemia and cerebral hemorrhage.

24. The composition of claim 20, wherein the isoquinoline derivative compound or a pharmaceutically acceptable salt thereof may be characterized by any one or more of the following:
a) specifically increasing mitophagy activity, not autophagy activity; and
b) not inducing mitochondrial dysfunction.

25. A method for preparing the isoquinoline derivative compound of claim 1, comprising the step (step 1) of adding palmatine represented by Chemical Formula 2 or berberine represented by Chemical Formula 3 to an organic solvent and reacting with a Lewis acid catalyst, as in Reaction Formula 1 below, to produce the isoquinoline derivative compound represented by Chemical Formula 1:

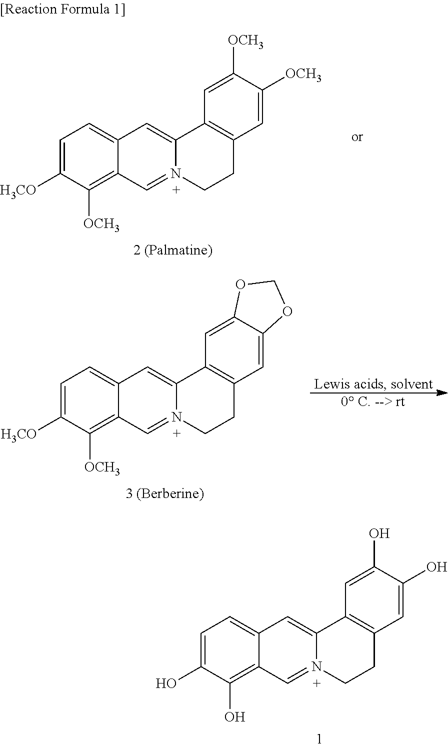

26. The method of claim 25, wherein the Lewis acid catalyst is any one or more selected from the group consisting of BF₃, BBr₃, AlF₃, AlCl₃, AlBr₃, TiCl₄, TiBr₄, TiI4, FeCl₃, FeCl₂, SnCl₂, SnCl₄, WCl₆, MoCl₅, SbCl₅, TeCl₂, ZnCl₂, Et₃Al, Et₂AlCl, EtAlCl₂, Et₃Al₂Cl₃, (i-Bu)₃Al, (i-Bu)₂AlCl, (i-Bu)AlCl₂, Me₄Sn, Et₄Sn, Bu₄Sn, and Bu₃SnCl.

27. The method of claim 25, wherein the organic solvent is any one or more selected from the group consisting of dimethyl sulfoxide, dimethyl formamide, acetone, tetrahydrofuran, benzene, toluene, ether, methanol, hexane, cyclohexane, pyridine, acetic acid, carbon tetrachloride, chloroform, dichloromethane, and water.

28. The method of claim 25, wherein the Lewis acid catalyst is added in an inert gas atmosphere.

29. The method of claim 25, wherein the reaction is stirred for to hours after adding the Lewis acid catalyst.
